(19) United States
(12) Patent Application Publication
(10) Pub. No.: US 2003/0232075 A1
(43) Pub. Date: Dec. 18, 2003

(54) Compositions for producing factor Xa

(75) Inventor: Gary L. Nelsestuen, St. Paul, MN (US)

Correspondence Address: FISH & RICHARDSON P.C., 3300 DAIN RAUSCHER PLAZA, 60 SOUTH SIXTH STREET, MINNEAPOLIS, MN, 55402, US

(73) Assignee: University of Minnesota, a Minnesota corporation

(21) Appl. No.: 10/430,844

(22) Filed: May 6, 2003

Related U.S. Application Data
(60) Provisional application No. 60/378,428, filed on May 6, 2002.

(51) Int. Cl. A61K 38/18; A61K 9/127; A61K 31/739

(52) U.S. Cl. 424/450; 514/12; 514/54

(57) ABSTRACT

Compositions that include tissue factor incorporated into lipid vesicles are described, as well as compositions that include enzymes other than factor VIIa that directly activate factor X to Xa in solution without directly supporting thrombin generation. Such compositions do not produce thrombogenic levels of thrombin in human patient and can be used to increase clot formation in patients in need thereof.

### CROSS REFERENCE TO RELATED APPLICATION

This application claims priority from U.S. Provisional Application Serial No. 60/378,428, filed on May 6, 2002.

### TECHNICAL FIELD

This invention relates to compositions for producing factor Xa, and more particularly, to compositions that produce factor Xa but do not produce thrombogenic levels of thrombin when administered to a human patient.

### BACKGROUND

Hemophilia is a bleeding disorder that most commonly arises as a genetic disorder when a male child inherits, from his mother, an X-chromosome containing a deficient factor VIII or factor IX gene. Factor VIII deficiencies account for approximately 80% of the genetic disorders, with factor IX deficiencies accounting for approximately 20% of the genetic disorders. Other proteins in the blood-clotting cascade can be deficient in both sexes, although these deficiencies account for only a small percentage of the cases. Hemophilia also can be ‘acquired’ as an autoimmune disease after the body begins to make antibodies to one of the blood clotting proteins. Factor VIII and von Willebrand's factor are the most common targets.

For treatment of standard hemophilia, the missing or deficient protein typically is replaced, a procedure termed factor replacement therapy. In many patients, including all patients with ‘acquired’ hemophilia, however, the body begins to make antibodies against the administered protein and factor replacement therapy fails. These antibodies are commonly referred to as ‘inhibitors’; hemophilia patients can be classified as having severe hemophilia with inhibitors or severe hemophilia without inhibitors.

Treatment options for individuals who develop inhibitors is limited and often of low efficacy and high cost. One option is alternative factor replacement, in which porcine factor VIII is used instead of human factor VIII. Obvious drawbacks of this therapy include antigenic response, which limits the number of times the therapy can be used. Another option is to bypass factor VIII and IX using, for example, prothrombin complex concentrate (PCC). PCC is a concentrated mixture of vitamin K-dependent proteins isolated from human blood. The mechanism of action of PCC is not known, although several theories have been proposed, including increasing zymogen levels in the blood (factors VII, IX, X and prothrombin) (Key, and Christie et al. (2002) Thromb. Haemost. In press 88:60-65), induction of tissue factor on the blood cell surface (Teitel et al. (1988) Thromb. Haemost. 60(2):226-229), and introduction of active clotting factors into the circulation (Sultan and Loyer (1993) J. Lab. Clin. Med. 121(3):444-452). In fact, some preparations of PCC are purposely activated to increase the level of active clotting factors in the preparation. Most preparations of PCC have specified levels of factor Xa activity. Thrombin is eliminated from all PCC preparations as this is the final product of the coagulation cascade (discussed in Thomas, W. R. U.S. Pat. No. 4,287,180) and will form thrombi wherever it is located.

A relatively new therapy for severe hemophilia with inhibitors is the use of high dose factor VIIa. A dose level of 90 μg/kg is suggested by the manufacturer, although much higher doses have been reported (up to 436 μg/kg for severe bleeds and 270 μg/kg for standard bleeding episodes). At 90 μg/kg every 3 hours, the concentration of factor VIIa in the plasma reaches 50 nM and should reach approximately 250 nM at a dosage of 436 μg/kg. As for PCC, the mechanism of action of factor VIIa is not known. Factor VIIa may directly activate factor X in a tissue factor dependent or independent mechanism. Tissue factor, the cofactor for factor VIIa, is an integral membrane protein found on the surface of some cells and is exposed during some cell activation steps or tissue damage. Results from factor VIIa and PCC therapy are inconsistent and treatment frequently fails. See, Lusher et al., (1998) Blood Coagul. Fibrinolysis, 9(2):119-28. As a result, patients may be treated in many different ways before hemostasis can be attained. Thus, there is a need for effective treatments of hemophilia and other clotting disorders that can occur with cancer and liver disease.

### SUMMARY

The invention is based on the discovery that tissue factor can be formulated in a manner that prevents thrombosis through subsequent action of the prothrombinase complex (factor Xa and Va) on a membrane surface. To prevent thrombogenic levels of thrombin from being generated, tissue factor can be reconstituted in vesicles with no acidic phospholipids and/or in vesicles containing phospholipids that prevent effective assembly of the prothrombinase complex (e.g., polyethylene glycol (PEG)-linked phospholipids). As a result, tissue factor formulated in such a manner can be used to treat hemophilia or other clotting disorders that may occur with cancer or liver disease. Without being bound to a particular mechanism, tissue factor formulated in such a manner can generate systemic levels of factor Xa throughout the bloodstream of a patient, thereby creating a coagulation-ready state. Alternatively, an enzyme, other than factor VIIa, that directly activates factor X to Xa in solution, in the absence of factors VIII or IX, can be used to create a coagulation-ready state in a patient. Therapies of the invention are superior to PCC and other therapies, which introduce factor Xa at the site of injection but are rapidly inhibited, resulting in uneven distribution of factor Xa in the circulation. Therapies of the invention also are superior to high dose factor VIIa therapy. While factor VIIa can achieve a coagulation-ready state through a similar mechanism, it is a poor enzyme without tissue factor, and as a consequence, high levels of factor VIIa are required to produce the coagulation ready state.

In one aspect, the invention features compositions and kits that include tissue factor, wherein the tissue factor is incorporated into lipid vesicles, and wherein the composition, upon administration to a human patient, produces non-thrombogenic levels of thrombin. The vesicles can include phospholipids or sphingolipids linked to a PEG polymer. The vesicles can include 0.5 to 50 mol % of the PEG polymer and the PEG polymer can have a molecular weight ranging from 500 to 80,000 (e.g., 20,000 to 40,000, 2,000 to 20,000, or 3,000 to 6,000). The phospholipids can include one or more phospholipids selected from the group consisting of phosphatidylcholine, phosphatidylethanolamine, and phosphatidylserine. The sphingolipids can include one or more sphingolipids selected from the group consisting of ceramide, sphingomyelins, cerebrosides, and gangliosides. The vesicles can include 0 to 20 mol % of acidic phospholipids or 5 to 50 mol % of glycolipids, and further can include phospholipids or sphingolipids linked to a polymer.

The invention also features a method for treating a clotting disorder in a patient. The method includes administering an amount of a composition to the patient effective to treat the clotting disorder, wherein the composition includes tissue factor incorporated into vesicles, and wherein the composition produces non-thrombogenic levels of thrombin in the patient. The method further can include administering a factor X polypeptide to the patient and/or administering a factor VIIa polypeptide to the patient.

In another aspect, the invention features a method for treating a clotting disorder in a patient that includes administering to the patient an amount of an enzyme, other than factor VIIa, effective for treating the clotting disorder. The enzyme directly activates factor X to factor Xa in solution. The enzyme can be a snake venom enzyme (e.g., the factor X activating enzyme from Russell's viper venom). The enzyme can be encapsulated in a lipid vesicle and/or linked to a PEG polymer.

In yet another aspect, the invention features an article of manufacture for treating a clotting disorder in a mammal. The article of manufacture includes a tissue factor composition, wherein the composition includes tissue factor incorporated into lipid vesicles, and wherein the tissue factor composition, upon administration to a human patient, produces non-thrombogenic levels of thrombin.

The invention also features compositions and kits that includes tissue factor incorporated into lipid vesicles, wherein the vesicles include phospholipids linked to a PEG polymer or sphingolipids linked to a PEG polymer. The vesicles can include 0.5 to 50 mol % of the PEG polymer and the PEG polymer can have a molecular weight ranging from 500 to 80,000 (e.g., 20,000 to 40,000, 2,000 to 20,000, or 3,000 to 6,000). The phospholipids can include one or more phospholipids selected from the group consisting of phosphatidylcholine, phosphatidylethanolamine, and phosphatidylserine. The sphingolipids can include one or more sphingolipids selected from the group consisting of ceramide, sphingomyelins, cerebrosides, and gangliosides.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### DESCRIPTION OF DRAWINGS

FIG. 1 is a log-log plot of coagulation time as a function of factor VIIa level.

FIG. 2 is a graph of the impact of various concentrations of factor X (40 to 630 nM) on factor VIIa (12.5 nM), low dose factor VIII (approximately 1% of normal factor VIII level in blood), and low dose tissue factor (20 nL of Innovin per mL of blood). Factor VIIa is represented by solid circles, tissue factor is represented by open circles, and factor VIII is represented by open squares.

FIG. 3 is a graph of the clotting time as a function of factor Xa concentration (5 to 160 pM).

FIG. 4 is a graph of the clotting time as a function of RVV-Xase enzyme concentration (40 to 400 fM).

FIG. 5 is a graph of the clotting time of hemophilic mouse blood or artificial hemophilic human blood as a function of several pro-coagulant reagents: Factor VIIa in mouse blood (solid circles with dashed line), factor VIIa in human blood (solid squares), factor Xa in human blood (open triangles), RVV-X in mouse blood (open squares) and RVV-X in human blood (solid circles with solid lines). Average and standard deviations (4 measurements) are shown.

FIG. 6 is a graph of the clotting time as a function of tissue factor concentration.

FIG. 7A is a graph of the clotting time stimulated by tissue factor reconstituted in pure PC vesicles and the impact of factor X and factor VIIa. The graph shows TF concentration as μL of this preparation per mL of blood. The following vesicles were tested: tissue factor-PC with no additions (open circles), TF-PC with addition of 310 nM factor X to whole blood (solid squares), and TF-PC with addition of 5 nM factor VIIa (solid circles). In all cases, the values represent the average and standard deviation of four measurements.

FIG. 7B is a graph of the clotting time stimulated by tissue factor reconstituted in pegylated vesicles and the ability of low factor VIIa to enhance reaction rates. Coagulation is shown for vesicles containing 20% PS (solid circles) without other additions to the blood. The other titrations are for vesicles of 0% PS (open circles) and 2% PS (solid squares), both with the addition of 5 nM factor VIIa to the clotting assay. In all cases, the values represent the average and standard deviation of four measurements.

FIG. 8 is a graph of the clotting time of different forms of tissue factor. The following forms of tissue factor were tested: soluble tissue factor (solid circles), full length tissue factor (open circles), full length tissue factor reconstituted in vesicles of 100% PC with (solid diamonds) and without (open diamonds) supplementation with 5 nM factor VIIa, and FL-TF reconstituted in vesicles of PC/PEG-PE (90/10) (solid triangles). Clotting times were recorded in the ACT-LR with artificial hemophilic human blood.

### DETAILED DESCRIPTION

In general, the invention features compositions containing tissue factor or soluble enzymes other than factor VIIa that are formulated in a manner such that, upon administration to a patient, thrombogenic levels of thrombin are not produced in the patient. Using the compositions described herein can be effective for increasing clot formation in patients, and as a result, can be used for treating patients with hemophilia or other clotting disorders in a low cost and effective manner, with reduced risk of thrombogenic complications for the patient. Assays provided by the invention can be used to detect factor Xa levels in the circulation of patients such that therapies can be adjusted or tailored for individual patients. Compositions of the invention can be combined with other therapies such as factor VIIa and/or factor X therapy.

Tissue Factor Compositions

Compositions of the invention can include tissue factor, an integral membrane protein found on the surface of certain cells (e.g., monocytes and cells of the blood vessel wall) that is exposed during certain cell activation steps or tissue damage. Tissue factor is the cofactor for factor VIIa and is considered to be active when associated with phospholipids. Native or wild-type human tissue factor can be used in compositions of the invention, as well as bovine, porcine, or ovine tissue factor. Preferably, native human tissue factor is used. In addition, tissue factor containing one or more amino acid substitutions, deletions, or insertions relative to wild-type tissue factor can be used.

Wild-type, human tissue factor is available commercially (e.g., Innovin, from Dade Behring, Inc., Deerfield, Ill.). Alternatively, tissue factor can be produced recombinantly using prokaryotic or eukaryotic host cells. See, for example, U.S. Pat. No. 6,261,803. The amino acid sequence of tissue factor can be found under GenBank Accession No. KFHU3. Recombinantly produced tissue factor can be purified using one or more chromatography steps, including gel chromatography, hydrophobic interaction chromatography, ion-exchange chromatography, or affinity chromatography. As tissue factor is typically associated with membrane, chromatography steps can be performed in the presence of a detergent, e.g., a nonionic detergent.

In compositions of the invention, tissue factor is incorporated into the membrane of lipid vesicles or liposomes such that it can interact with biomolecules surrounding the vesicle or liposome. As used herein, the terms “lipid vesicles” and “liposomes” are used interchangeably, and refer to both unilamellar and multilamellar vesicles and aggregates. The lipid vesicles can be composed of phospholipids such as phosphatidylcholine (PC), phosphatidylethanolamine (PE), phosphatidylserine (PS), and combinations thereof, and sphingolipids such as ceramide, sphingomyelins, cerebrosides, gangliosides, and combinations thereof. Reconstituting integral membrane proteins such as tissue factor in lipid vesicles can be accomplished by several methods, including detergent dialysis. See, for example, Mimms et al., (1981) Biochemistry 20:833-840. During detergent dialysis, the protein and lipid are mixed in a detergent that is subsequently dialyzed away. The lipids remain inside the dialysis membrane since they are in a micelle and gradually fuse into bilayer membranes as the detergent level declines. The protein also is made soluble by the detergent until its level declines, whereupon the hydrophobic membrane-associated region of the protein becomes incorporated into the lipid bilayer. Non-limiting examples of detergent that can be used for detergent dialysis include octylglucoside and deoxycholate.

The ratio of tissue factor to lipid in the vesicle can vary from 1:100 to 1:10,000 (w/w). Since the different phospholipid classes have relatively similar molecular weights, a weight ratio of PS/PC of 20/80 approximately equals a molar ratio of 20/80. In one series of experiments, a ratio of 1:1000 (w/w, tissue factor:phospholipid) provided the maximum activity per mg of tissue factor at the lowest level of phospholipid. For standard phospholipids of molecular weight 750, a 1:1000 weight ratio corresponds to approximately 1 gram of tissue factor per 1.33 moles of phospholipid.

As described in Example 5, tissue factor in lipid vesicles becomes more potent at a more rapid rate than other coagulation factors, which narrows the useful dosage range and provides increased potential to overdose and cause thrombosis, both of which are undesirable for pro-coagulant therapy. Without being bound to a particular mechanism, tissue factor and endogenous factor VIIa may produce factor Xa and the added membrane component (from the vesicle) may support subsequent reactions of the clotting cascade, such as factor Xa-factor Va activation of prothrombin to thrombin.

To prevent thrombin production, tissue factor can be presented as a composition that prevents direct support of thrombin production. In such compositions, tissue factor can be associated with its naturally occurring lipid as long as the composition prevents direct support of thrombin production. For example, PEG-linked phospho- or sphingolipids can be used (e.g., 0.5 to 50 mol %) to prevent direct support of thrombin production. PEG-modified lipids are available commercially from companies such as Avanti Polar Lipids or Shearwater Polymers, Inc. Suitable PEG polymers can have a molecular weight ranging from 500 to 80,000 (e.g., 20,000 to 40,000, 2,000 to 20,000, or 3,000 to 6,000). The molar percentage of PEG-modified lipids that can be incorporated into the vesicle depends on the size of the PEG polymer. In general, the smaller the molecular weight of the PEG polymer, the higher the percentage of PEG-modified lipids that can be incorporated into the lipid vesicle. For example, if a PEG polymer has a MW of 500, up to 40 mol % (e.g., 30 to 40 mol %) of PEG-modified lipids can be incorporated into the vesicle. If a PEG polymer has a MW of 80,000, approximately 0.5 to 5 mol % of PEG-modified lipids can be incorporated into the vesicle. The PEG moiety can be attached to the headgroup of the lipid (e.g., PE), thereby creating a negatively charged phospholipid. Alternatively, a PEG moiety can be attached to the headgroup of a sphingolipid such as ceramide, thereby creating a neutral membrane lipid molecule. Both forms are available from commercial sources. Preferably, the PEG-modified lipids are in the fluid phase at 37° C. (e.g., dioleolyl-PE-PEG).

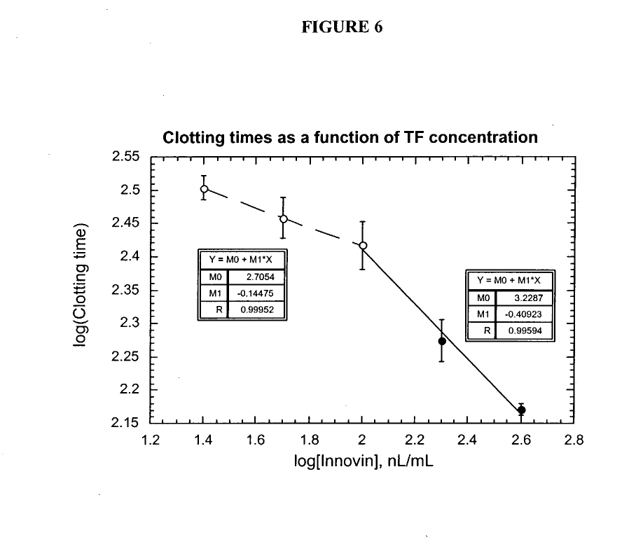

Incorporating PEG-lipids into vesicles interferes with fusion of the vesicle to blood cells and removal of the vesicle from circulation, which can increase circulation lifetime. Circulation half-lives of days have been reported for vesicles containing 10% PEG-5000 (Phillips and Klipper et al. (1999) J. Pharmacol. Exp. Ther. 288:665-670). As described herein, tissue factor in vesicles containing 10% PEG-5000 (10 mol percent, the molecular weight of the PEG was 5000) gave a desirable linear dose-response relationship (FIG. 6), mimicking the outcome for other clotting factors. PEG may sterically inhibit the assembly of Xa with Va on the membrane surface and the subsequent conversion of prothrombin to thrombin, thereby preventing thrombin production on these tissue factor-containing vesicles. Factor Xa can be produced in a less efficient reaction and can be released into the blood, where, despite it short half-life, it will be available to bind to damaged cells or activated platelets, the common recognition for coagulation.

To minimize direct support of thrombin production, tissue factor also can be incorporated in lipid vesicles with minimal amounts of acidic phospholipids (e.g., 0 to 20 mol % of PS, phosphatidylglycerol, or phosphatidic acid). For example, tissue factor can be in a liposome containing 0-20 mol % acidic phospholipid, with the remaining balance containing any neutral lipid. Polysaccharides or any other non-antigenic material that restricts access of prothrombinase components (Factors Xa and Va) also can be used. For example, 5 to 50 mol % glycolipids can be used. In some embodiments, compositions of the invention can include tissue factor incorporated into vesicles that contain PEG-modified phospholipids (5 to 50 mol %) and minimal amounts of acidic phospholipids (e.g., 1 to 10 mol %).

To assess if a composition is non-thrombin generating, the following plasma blood-clotting test can be used. Clotting reactions can be started by adding 112.5 μL of 0.05 M Tris buffer containing 100 mM NaCl and 6.7 mM CaCl₂ to 37.5 μl of factor VII-deficient, citrated human plasma (e.g., Sigma Chemical Company, St. Louis, Mo.) and incubating at 37° C. The time required for the solution to coagulate can be measured by visual evaluation and the hand tilt method. To stimulate coagulation on any available membrane surface, 2 nM factor Xa (Enzyme Research Laboratories) can be included in the solution. Since the plasma itself contains a small amount of lipid, the control has a clotting time (approximately 38.2±0.9 sec). Overall, non-thrombogenic vesicles containing tissue factor are defined as those that produce less than a 1 second change in clotting time in a comparable plasma assay where the vesicles are present at 100-times the level that produces the therapeutic coagulation time in the whole blood clotting assay. To provide the proper evaluation, it may be necessary to screen the factor VII-deficient plasmas to ensure a background clotting time that is comparable to the value presented herein. As described in Example 6, adding 5 μg of vesicles (PS/PC/PE-PEG-5000, 20/70/10 mol ratio) gave a clotting time of 37.7±1.2 sec, a non-detectable change in clotting time showing undetectable thrombin production. This lipid concentration is 100-fold higher than that which produces a clotting time of 310 seconds in the assay system described in FIG. 6. In an experiment using 5 μg of PS/PC/PE-PEG-2000 (20/70/10) vesicles, a clotting time of 34.9±0.8 seconds was detected, which was significantly shorter than the clotting time of the control. Consequently, the latter vesicles supported thrombin production by factor Xa and would not be suitable for use in therapy. Addition of 1 μg of PS/PC (20/80) vesicles to this assay produced a clotting time of 14.5±0.2 seconds.

Lipid vesicles containing tissue factor can be lyophilized using known techniques and stored for later use. A cryopreservation agent such as one or more carbohydrates (e.g., trehalose, maltose, lactose, glucose, or mannitol) can be included during the lyophilization. Lyophilized vesicles can be reconstituted in buffers suitable for administration to a patient, as described below.

Enzyme Compositions

Soluble enzymes, other than factor VIIa, that directly produce factor Xa in the absence of factors VIII or IX, also can be used to regulate clotting. Non-limiting examples of suitable enzymes include enzymes from snake venom such as an enzyme from Russell's Viper Venom (RVV-Xase). The antigenicity of RVV-Xase may elicit an immune response in patients, limiting the length of time that it can be used in patients. To minimize antigenic response to RVV-Xase, a PEG polymer can be attached to the enzyme, e.g., at exposed lysine residues. Modification of proteins with PEG can extend their lifetimes in the blood circulation and lower their antigenic properties. A number of amino or sulfhydryl-reacting forms of PEG polymers are available from commercial sources such as Shearwater Polymers, Inc. The degree of modification can be monitored to balance the goal of reducing antigenic activity vs. retention of enzyme activity. However, such derivatives can prove very useful when using a foreign protein in a therapeutic role.

Alternatively, the enzyme can be encapsulated in lipid vesicles that allow slow enzyme release. The latter technique may extend the time of therapy and allow extended efficacy of RVV-Xase even after development of an immune response. Protein encapsulation into lipid vesicles (e.g., phospholipid vesicles) can be accomplished by several suitable techniques. For example, detergent dialysis can be used as outlined above. Alternatively, phospholipids can be dried in a glass tube and dispersed in buffer containing the protein. Monobilayer vesicles can then be generated by freeze-thaw of the solution and subsequent extrusion of the vesicles through porous membranes. See, for example, Malinski and Nelsestuen (1989) Biochemistry 28:61-70.

Pharmaceutical Compositions

Compositions of the invention can be formulated into pharmaceutical compositions by admixture with pharmaceutically acceptable non-toxic excipients or carriers, and used to regulate coagulation in vivo. Generally, the composition can be administered by any suitable route of administration, including orally, transdermally, intravenously, subcutaneously, intramuscularly, intraocularly, intraperitoneally, intrarectally, intravaginally, intranasally, intragastrically, intratracheally, intrapulmonarily, or any combination thereof. Compositions can be prepared for parenteral administration, particularly in the form of liquid solutions or suspensions in aqueous physiological buffer solutions; for oral administration, particularly in the form of tablets or capsules; or for intranasal administration, particularly in the form of powders, nasal drops, or aerosols. Parenteral administration is particularly useful. Compositions for other routes of administration may be prepared as desired using standard methods.

Formulations for parenteral administration may contain as common excipients sterile water or saline, polyalkylene glycols such as polyethylene glycol, vegetable oils, hydrogenated naphthalenes, and the like. In particular, biocompatible, biodegradable lactide polymer, lactide/glycolide copolymer, or polyoxethylene-polyoxypropylene copolymers are examples of excipients for controlling the release of a composition in vivo. Other suitable parenteral delivery systems include ethylene-vinyl acetate copolymer particles, osmotic pumps, implantable infusion systems, and liposomes. Formulations for parenteral administration also may include glycocholate for buccal administration.

Methods of Increasing Clot Formation

Compositions of the invention can be administered to patients in need thereof (e.g., hemophilia patients, cancer patients, patients with liver disease, or other patients with severe bleeding such as trauma patients). A patient's clotting activity can be assessed before administering a composition to provide a baseline clotting time. Such an assessment also allows the amount of tissue factor or soluble enzyme in the composition to be tailored to the particular patient.

The dosage of composition required to increase clot formation in the mammal depends on the route of administration, the nature of the composition, the subject's size, weight, surface area, age, and sex, other drugs being concurrently administered, and the judgment of the attending physician. Wide variations in the needed dosage are to be expected in view of the variety of compositions that can be produced (e.g., the nature of phospholipids and/or degree of PEG-modified lipids), the variety of subjects to which the composition can be administered, and the differing efficacies of various routes of administration. In general, the clotting activity of tissue factor and/or enzymes that support factor Xa generation should be in the range of 0.5 to 50 units (e.g., 0.5 to 2.5, 2.5 to 10, or 10 to 50 units) per mL of patient's blood, where one unit of activity is defined as the amount needed to produce a clotting time of 370 seconds in one mL of normal response blood (NRB) using the whole blood clotting test described in Example 1. NRB is defined as a sample of factor VIII-deficient blood in which 50 nM wild type factor VIIa produces a clotting time of 370 seconds.

After a composition is administered to a patient, clotting time can be monitored to evaluate the therapy. It may be desirable to combine factor VIIa therapy and/or factor X therapy with the compositions of the invention. Adding factor VIIa to the therapy regimen may lower the amount of tissue factor that is needed since it can displace endogenous factor VII and give a more consistent and potent result. Endogenously, factor VIIa is a very small portion of total factor VII in the circulation system, typically 1% or 0.1 nM. Thus, an amount of factor VIIa can be administered to the patient that will generate 0.5-10 nM of factor VIIa in the blood, which can create a stronger and more consistent patient response. One unit of factor VII is the amount of factor VIIa needed to produce a clotting time of 370 seconds in one mL of blood using the blood clotting assay of Example 1; one unit of factor VIIa corresponds to 50 nM factor VIIa. The short circulation half time of factor VIIa (2 to 3 hours) would also provide rapid reversal of coagulation activity in the case of overdose.

Native or wild-type human factor VIIa polypeptide can be used, as well as modified factor VIIa that contains one or more amino acid substitutions, deletions, or insertions relative to wild-type factor VII. Factor VIIa having enhanced membrane binding affinity and/or activity is particularly useful. See, for example, the factor VIIa polypeptides of U.S. Pat. No. 6,017,882 and Shah et al. (1998) Proc. Natl. Acad. Sci. USA 95:4229-4234 (e.g., factor VIIa containing a glutamine at position 10 and a glutamic acid residue at position 32). If a factor VIIa polypeptide having enhanced membrane binding affinity and/or activity is used, a lowered dosage of factor VIIa can be used.

As indicated above, factor X therapy can be combined with the methods of the invention. Suitable amounts of factor X can be used to generate a level of 30 to 500 nM in whole blood or about 130 to 2000 μg/kg body weight. Typically, 0.5 to 50 units of factor X can be administered. One unit of a factor Xa is the amount that will give a clotting time of 370 seconds in the clotting assay described in Example 1. Supplementing factor X during therapy can replace any factor X that is consumed by the tissue factor-factor VIIa or factor X activating enzyme. Native or wild-type human factor X can be used, as well as modified factor X containing one or more amino acid substitutions, deletions, insertions relative to wild-type factor X. Particularly useful modified factor X polypeptides having enhanced membrane binding affinity and activity are described in WO 00/66753.

For chronic management of clotting disorders, the clotting assay described herein can be used to set a range of acceptable dosages for the patient's home therapy since individuals tend to give similar results over time. A patient's blood can be tested in vitro by adding a composition of the invention to a sample of the patient's blood and assessing clotting time. In this way, a specific clotting time can be targeted for all individuals rather than a single dosage for all patients, which is the current practice.

Articles of Manufacture

Compositions described herein can be combined with packaging materials and sold as articles of manufacture or kits. Components and methods for producing articles of manufactures are well known. The articles of manufacture may combine one or more compositions described herein. In addition, the articles of manufacture may further include one or more of the following: sterile water, pharmaceutical carriers, buffers, antibodies (e.g., anti-factor VIII:C or anti-factor IX), calcium chelators, calcium containing solutions, factor VIIa, factor X, and/or other useful reagents for treating clotting disorders. A label or instructions describing how tissue factor or a soluble enzyme other than factor VIIa can be used for treatment of clotting disorders (e.g., for increasing clot formation in a hemophiliac) may be included in such kits. The compositions or individual components may be provided in a pre-packaged form in quantities sufficient for single or multiple administrations.

The invention will be further described in the following examples, which do not limit the scope of the invention described in the claims.

### EXAMPLES

The following materials were used unless otherwise indicated. Recombinant factor VIIa (NovoSeven®) was obtained from Novo Nordisk, Princeton, N.J. Purified factor Xa and RVV-Xase were obtained from Enzyme Research Laboratories, Inc, South Bend, Ind. Tissue factor (TF) was obtained from Dade Behring, Inc. (Innovin, Deerfield, Ill.). Anti-human factor VIII antibodies were obtained from Affinity Biologicals, Inc., Hamilton, Ontario. PEG-linked phospholipids were obtained from Shearwater Polymers, Inc. (Huntsville, Ala.) and from Avanti Polar Lipids (Alabaster, Ala.).

### Example 1

In Vitro Clotting Assay:

Whole blood was analyzed in the Hemochron Jr. Signature Microcoagulation instrument (International Technidyne, Inc.) using the ACT-low range (LR) cuvette. See also Nelsestuen et al. (2001) Abstract P1397 from the XVIII Congress of the International Society of Thrombosis and Haemostasis. The ACT-LR cuvette contains celite to active the intrinsic coagulation cascade and no added phospholipid. Celite is not necessary to perform the assay. With this instrument and cuvette, normal blood coagulates in 160±20 seconds, blood from severe hemophiliacs coagulates in >400 seconds, and blood from patients with 1% factor VIII or IX coagulates with an average time of 357 seconds.

To perform the assay, blood was drawn from a normal individual and nine volumes of the blood mixed with 1 volume of 0.1 M sodium citrate (or 1 volume of another calcium chelator). The samples were stored in 14 mL plastic conical tip tubes with screw top caps, each containing about 2 mL of blood. Affinity-purified anti-human factor VIII:C antibodies were added to the chelated blood in an amount sufficient to block all detectable factor VIII:C. This amount was estimated by determining if clotting time of the blood increased to greater than 400 seconds. Typically, 6-8 μg of anti-human factor VIII:C (Affinity Biologicals, Inc., Hamilton, Ontario) are added per mL of blood.

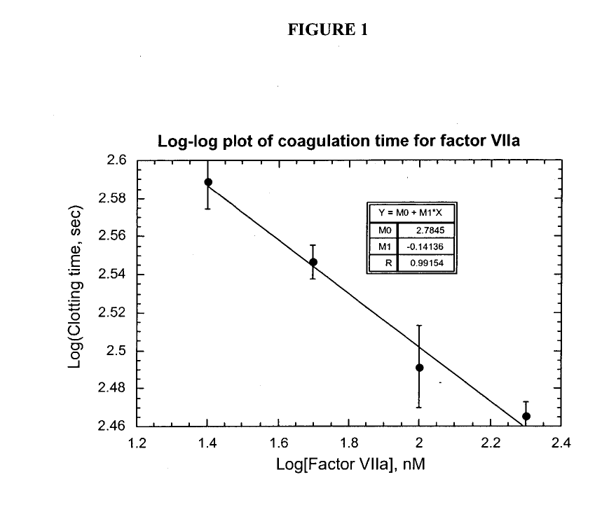

After incubating the blood and 6 μg of anti-human factor VIII:C antibody for about an hour at room temperature, the clotting assay was performed. The cells in the tube were suspended by tipping the tube about five or six times. The blood was re-calcified by mixing 0.1 mL of blood with 2.5 μL of 0.4 M CaCl₂ in a small plastic tube. Factor VIIa was added to the tube (1.2 to 2.4 nM) and mixed, then transferred to an LR-cuvette. Clotting time was measured by the Hemochron Jr. instrument. Data in FIG. 1 are plotted as log (Clotting time) vs. log[titrant]. The slope of the curve was −0.14. Error bars represent 2 standard deviations for the data obtained for one individual over a 2-year period. This assay is useful for monitoring clotting times as it encompasses therapeutic levels of factor VIIa (MW=50,000), which are 90 to 436 μg/kg body weight. Given approximately 75 mL of blood per kg body weight, therapeutic dosages will produce 25-125 nM factor VIIa in whole blood and approximately twice this level in plasma.

### Example 2

Impact of Factor X on Coagulation:

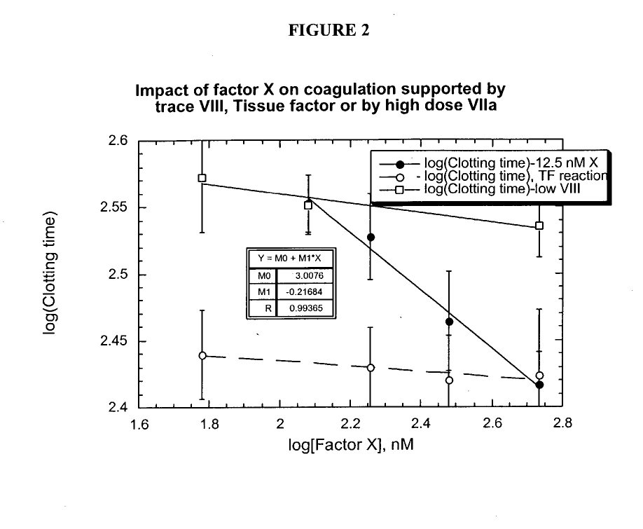

The impact of factor X concentration on coagulation was assessed using a trace amount of factor VIII, tissue factor (20 nL of Innovin per mL of blood), or high dose factor VIIa (12.5 nM). As indicated in FIG. 2, increased levels of factor X increased the efficacy of factor VIIa, but did not impact coagulation supported by either low levels of factor VIII or by low levels of TF-VIIa that were introduced into the blood. The result for factor X contrasted with that for prothrombin. Addition of prothrombin to blood at a level that doubled its normal concentration did not have a significant impact on the clotting time in the ACT-LR (data not shown).

It is possible that the increased function of factor VIIa following PCC therapy is a result of increased factor X levels. Individuals on PCC therapy have been found to have up to 5 times the normal factor X level in their circulation systems. For example, the level of factor X in the plasma of a patient who had received PCC every 12 hours for one week was 5-fold higher than that of normal plasma when blood was drawn 9 hours post PCC administration. Twenty hours after switching to 24-hour PCC administration, the level of factor X in the plasma of this individual was 3.4-fold higher than normal plasma. Four-fold higher factor X was found in another patient immediately after PCC administration (24-hour schedule). The same individual showed approximately 3-fold higher factor X level immediately before this treatment. The favorable impact of high levels of factor X on VIIa therapy points to a mechanism that differs from TF-dependent factor VIIa action or enhancement of coagulation that is based on trace factor VIII levels in the blood. A reaction with high Km may explain synergy of PCC and factor VIIa therapies in vivo.

### Example 3

Solution-Phase Activation of Factor X:

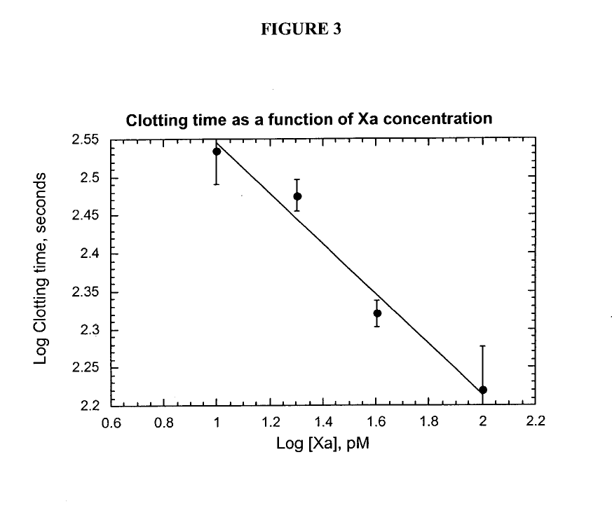

To test for solution-phase activation of factor X, the levels of factor Xa needed to support the coagulation activity created by high dose factor VIIa were determined. Solution-phase activation of factor X by factor VIIa was tested by mixing the proteins in buffer containing 5 mM calcium (pH 7.5). Appearance of factor Xa in the solution was detected by addition of an aliquot of the activation mixture to the whole blood clotting test. Indeed, factor Xa activity did increase in a reaction containing 200 nM factor VIIa, but not in a control reaction containing only factor X. Clotting times (as determined in Example 1) upon addition of this activation mixture to factor VIII-deficient blood indicated a factor Xa concentration of approximately 700 pM in the activation reaction after a 3 hour incubation at 37° C. Factor Xa activity was determined by comparison to a standard curve created by addition of factor Xa directly to factor VIII-deficient blood (FIG. 3). This correspond to a rate of Factor X activation of approximately 3.6 pM per minute. The clotting time for the highest dose of factor VIIa (436 μg/kg or 125 nM in whole blood, clotting time of 310 seconds, FIG. 1) required only about 15 pM factor Xa. The average level of factor Xa over the coagulation time would actually be lower than the added amount of added factor Xa since it is rapidly inactivated in whole blood. For example, 40 pM factor Xa, incubated in whole, citrated blood at 37° C. was inhibited by more than 90% in 5 minutes (data not shown), indicating a half-life for factor Xa in whole blood of only about 1.6 minutes. Consequently, addition of 15 pM factor Xa in the clotting test, as in FIG. 3, would provide an average level of about 7 pM factor 20 Xa during the 5.2 minutes required to form a clot.

Given a half-life of 1.6 minutes, the steady state level of factor Xa produced by 200 nM factor VIIa, would be approximately 8.4 pM. This is almost equal to the average level of factor Xa needed to produce a clotting time of 310 seconds, the response time at 125 nM factor VIIa. In any event, this level of factor Xa would impact on the clotting times observed at therapeutic doses of factor VIIa (50-250 nM/mL of whole blood or 50-250 nM in plasma). Similar reactions performed with the high affinity mutant of factor VIIa (QE-VIIa) showed nearly identical rates of factor X activation in solution. This suggested that factor X activation was truly solution-based and was not influenced by minor contamination by phospholipid vesicles, which would distinguish the outcome for wild type vs. mutant VIIa.

Importantly, this rate of factor X consumption would not deplete factor X in the circulation system. A rate of 5 pM/min in whole blood would consume only 7 nM factor X in a 24 hour period, about 10% of the factor X level in normal whole blood. A smaller decline would actually be observed as factor X is constantly being produced. Solution-based activation was also very low when compared with the reported reaction on activated platelet surfaces where 1-2 nM Xa was produced per hour at 50 nM VIIa and 120 nM factor X. This is a rate of about 25 pM per minute at a factor VIIa level of 1/4 that used for the solution-based activation described here. Thus, despite very low and often undetected action, it is possible that a significant portion of wild type factor VIIa activity arises from solution-based action.

Overall, this result showed that therapeutic doses of factor VIIa do in fact produce detectable levels of factor Xa from solution-phase activation and that any membrane-associated reaction will enhance the concentration of factor Xa. The mechanism of action of factor VIIa may consist of constant low levels of factor Xa that produce a coagulation-ready state. A part of the Xa may arise from solution-phase activation of factor X. Upon exposure of the appropriate membrane surface, this factor Xa will bind and initiate the remaining coagulation cascade, thereby bypassing the steps involving factors VIII or IX. If factor Xa is the only active enzyme in the blood, unwanted thrombosis may be avoided, thereby providing a safe mechanism to induce coagulation. That the levels of factor Xa detected in this study can be effective is supported by a recent study that examined a model system for coagulation where pM factor Xa appeared very early in coagulation and was important for an extended time of coagulation (Hockin et al., (2002) J. Biol. Chem. 227(21):18322-33).

### Example 4

Blood Clotting as a Function of RVV-Vase:

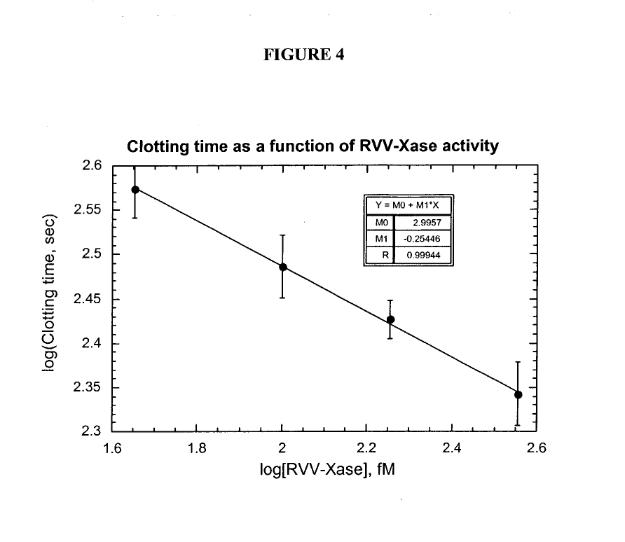

Blood clotting was assessed as in Example 1, in the presence of 40 to 400 fM of RVV-Xase. The results are shown in FIG. 4. About 90 fM enzyme provided the clotting times produced by 125 nM factor VIIa (the peak concentration of factor VIIa in whole blood at the highest reported dose). This corresponds to about 30 ng of RVV-Xase for a dose level in a typical adult. Thus, very low levels of RVV-Xase enzyme were needed, making this a very economical method of producing low levels of factor Xa in the circulation.

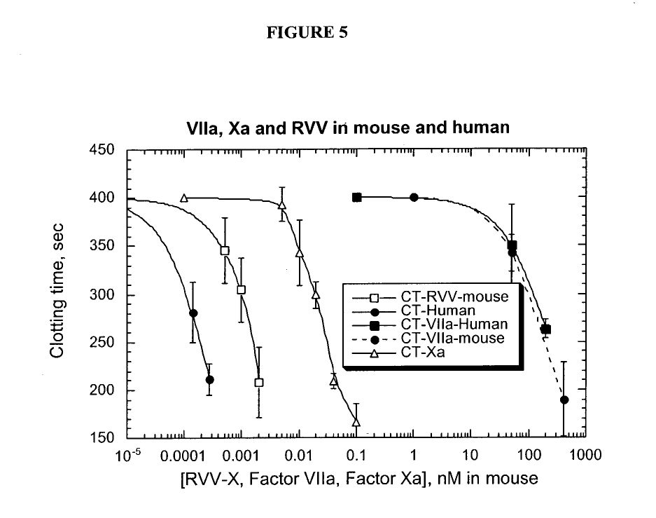

A titration of RVV-X vs. clotting time in factor VIII-depleted human blood or hemophilic mouse blood is shown in FIG. 5. Human blood was about 8-fold more sensitive to RVV-X than mouse blood. However, both required sub-picomolar levels to support coagulation times of 200 nM factor VIIa (FIG. 5). In human blood, the level of RVV-X needed for a given clotting time was approximately 0.01 times the factor Xa level.

### Example 5

Coagulation Time as a Function of Added Tissue Factor:

Clotting time was assessed as in Example 1, in the presence of tissue factor. In this experiment, tissue factor was supplied as Innovin, which contains approximately 40 μg of phospholipid per mL and is approximately 1 nM in TF. As indicated in FIG. 6, tissue factor is very effective as only about 28 nL of Innovin were required per mL of blood (150 μL or 6 ng of protein for a complete dose to a typical adult) was required to produce a clotting time of 310 seconds, the value expected at 436 μg of factor VIIa/kg body weight. Similar results were obtained for TF that was reconstituted in vesicles of phosphatidylserine (PS):phosphatidylcholine (PC) 20:80, at a 1:1000 weight ratio (approximately 1 gram of tissue factor per 1.33 moles of phospholipid) using the detergent dialysis method and octylglucoside.

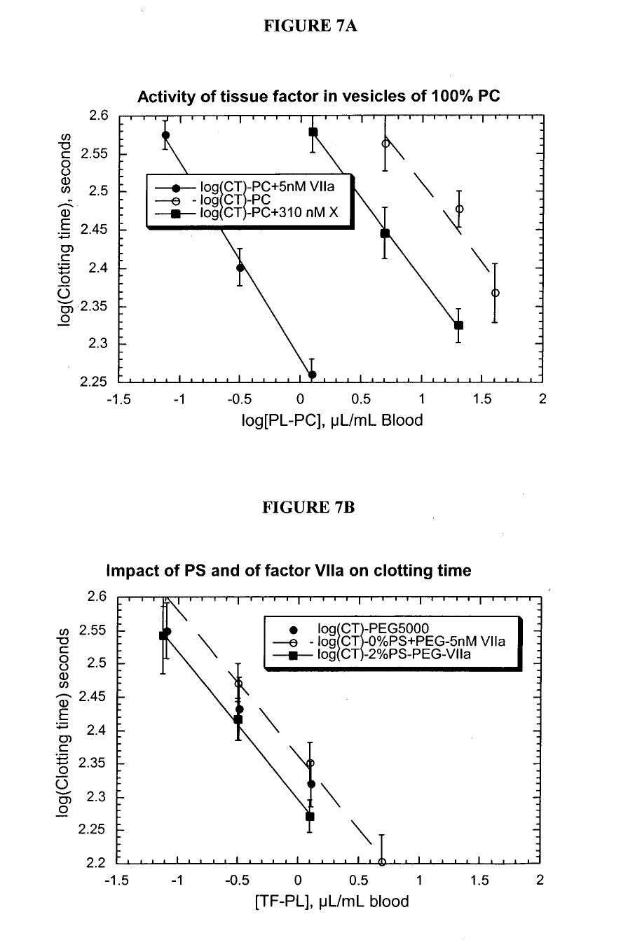

An adverse aspect of the tissue factor titration in FIG. 5 was downward curvature of the plot. In effect, TF became more potent at a more rapid rate than any other coagulation factor (compare FIGS. 1-4). Downward curvature may narrow the desired dosage range of tissue factor and would provide increased potential to overdose and cause thrombosis. Downward curvature may arise from direct thrombin production on the Innovin membrane surface. To minimize this adverse property, coagulation also was assessed with tissue factor preparations in the presence and absence of PS, factor X, and factor VIIa. The coagulation assay was performed as described in Example 1, with the addition of tissue factor (1.25 μg), which was reconstituted in phospholipid (equivalent to 1.0 mg) and dialyzed to a final volume of 0.4 mL. The results are presented in FIG. 7A. The tissue factor preparations and other additions include: pure PC with no additions (open circles), pure PC with addition of 310 nM factor X to the whole blood (solid squares), and pure PC with addition of 5 nM factor VIIa (solid circles). Pegylated phospholipid preparations also were tested using vesicles containing 10 mol % of PE-PEG-5000 (FIG. 7B). Vesicles containing PS/PC/PE-PEG5000 (20/70/10, mol ratio, the total molar amount of phospholipid was the same as that of the pure PC preparation) were tested (solid circles). Also shown is tissue factor reconstituted in vesicles of PC/PE-PEG5000 (90/10, no PS) with 5 nM factor VIIa added (open circles) as well as PS/PC/PE-PEG5000 (2/88/10) with 5 nM factor VIIa added (solid squares). Neither of the latter preparations gave measurable clotting times without added factor VIIa.

As indicated in FIG. 7A and FIG. 7B, adding low levels of factor VIIa had a large, positive impact on activity of the non-thrombogenic vesicles. This is expected since endogenous factor VII is about 10 nM in the blood, with factor VIIa, the active enzyme, being only about 1% or 0.1 nM. Addition of 5 nM factor VIIa increased the level of the active tissue factor-factor VIIa complex. Added factor X also shows an impact on these vesicles. This is also expected since pure PC has low affinity for factor X, thereby providing a low affinity for interaction of factor X with the tissue factor-factor VIIa enzyme. The enhancement by low levels of added factor VIIa is extremely large for the most non-thrombogenic vesicles. It appeared that factor X activation activity can be regulated very closely by addition of small amounts of factor VIIa. This may offer additional advantage for control of coagulation but at low cost.

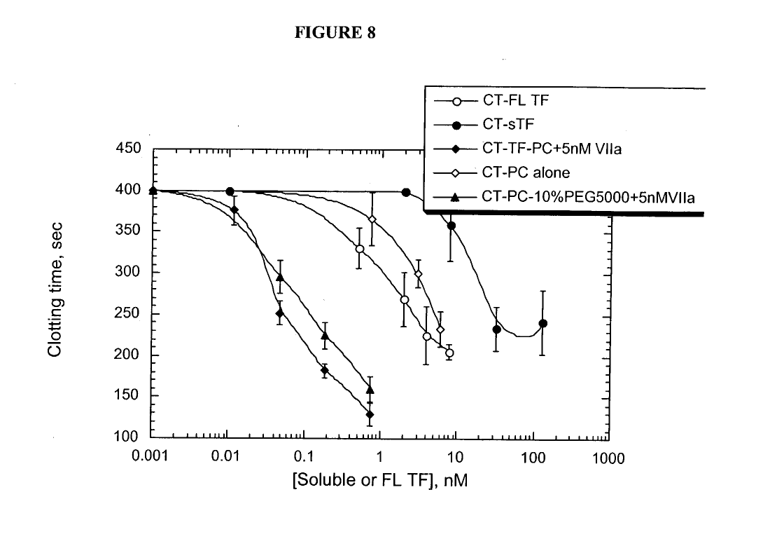

There are a number of ways that TF can be administered without a thrombogenic membrane component. FIG. 8 shows results for the apoproteins, soluble (s) TF and full-length (FL)-TF, as well as for membrane-bound FL-TF in pure PC vesicles and vesicles containing PEG-PE. Neither of these membranes should support thrombin production. A number of other combinations were tested. For example, including low levels of PS (2 to 10 percent) in membranes containing PEG-PE produced a more effective agent. While PS-containing membranes are normally thrombogenic, the presence of PEG-PE prevented thrombin production, as detected by the ability of these membranes to function as a thromboplastin in the PT assay.

### Example 6

In Vivo Safety of RVV-Xase:

An important property of high dose factor VIIa therapy is its apparent safety with few adverse reactions reported. If constitutive activation of factor Xa were the mechanism of factor VIIa action, direct activation of factor X by enzymes such as RVV-X should also be very safe when administered intravenously. In fact, RVV-X was very safe, even at concentrations of over 1000-times the dose needed to mimic therapeutic levels of factor VIIa in vitro (Table 1). Very few adverse reactions were detected at these extreme concentrations, in either wild type mice or in hemophilic mice. Limited evidence may suggest higher toxicity in older animals (Table 1). At the extreme dose in the animal, results from solution phase activation of factor X suggest that 100% of the plasma factor X should be activated in less than 1 minute. Overall, the safety of intravenous RVV-X administration appears to correlate well with the observed safety of high dose factor VIIa in vivo.

TABLE 1
Safety of Intravascular RVV-X Injection

| | | | | Adverse |
|---|---|---|---|---|
| Strain of | Age | Dose | Concentration | reactions^{a}/total |
| Mouse | (Weeks) | (μg/kg) | in whole blood | animals |
| C57 Black | 8 ± 1 | 10 | 1.25 nM | 0/4 |
| C57 Black | 8 ± 1 | 1.0 | 0.12 | 0/4 |
| C57 Black | 8 ± 1 | 0.1 | 0.012 | 0/4 |
| E16 | 8 ± 1 | 16 | 2.0 | 1/3 |
| E16 | 16 | 10 | 1.2 | 0/4 |
| BalbC | 37 | 16 | 2.0 | 1/2 |
| BalbC | 37 | 1.6 | 0.2 | 1/4 |
| Overall | | | | 3/25 |

^{a}Adverse response includes death or impairment of movement

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising tissue factor incorporated into lipid vesicles, and wherein said composition, upon administration to a human patient, produces non-thrombogenic levels of thrombin.

2. The composition of claim 1, wherein said vesicles comprise phospholipids or sphingolipids linked to a polyethylene glycol (PEG) polymer.

3. The composition of claim 2, wherein said vesicles contain 0.5 to 50 mol % of said PEG polymer.

4. The composition of claim 2, wherein said PEG polymer has a molecular weight ranging from 500 to 80,000.

5. The composition of claim 2, wherein said PEG polymer has a molecular weight ranging from 20,000 to 40,000.

6. The composition of claim 2, wherein said PEG polymer has a molecular weight ranging from 2,000 to 20,000.

7. The composition of claim 2, wherein said PEG polymer has a molecular weight ranging from 3,000 to 6,000.

8. The composition of claim 2, wherein said phospholipids comprise one or more phospholipids selected from the group consisting of phosphatidylcholine, phosphatidylethanolamine, and phosphatidylserine.

9. The composition of claim 2, wherein said sphingolipids comprise one or more sphingolipids selected from the group consisting of ceramide, sphingomyelins, cerebrosides, and gangliosides.

10. The composition of claim 1, wherein said vesicles contain 0 to 20 mol % of acidic phospholipids.

11. The composition of claim 1, wherein said vesicles contain from 5 to 50 mol % of glycolipids.

12. The composition of claim 10, wherein said vesicles further comprise phospholipids or sphingolipids linked to a polymer.

13. A method for treating a clotting disorder in a patient, said method comprising administering an amount of a composition to said patient effective to treat said clotting disorder, wherein said composition comprises tissue factor incorporated into lipid vesicles, wherein said composition produces non-thrombogenic levels of thrombin in said patient.

14. The method of claim 13, said method further comprising administering a factor X polypeptide to said patient.

15. The method of claim 13, said method further comprising administering a factor VIIa polypeptide to said patient.

16. The method of claim 13, said method further comprising administering a factor X polypeptide and a factor VIIa polypeptide to said patient.

17. A method for treating a clotting disorder in a patient, said method comprising administering to said patient an amount of an enzyme, other than factor VIIa, effective for treating said clotting disorder, wherein said enzyme directly activates factor X to factor Xa in solution.

18. The method of claim 17, where said enzyme is a snake venom enzyme.

19. The method of claim 18, wherein said enzyme is the factor X activating enzyme from Russell's viper venom.

20. The method of claim 17, wherein said enzyme is encapsulated in a lipid vesicle.

21. The method of claim 17, wherein said enzyme is linked to a PEG polymer.

22. An article of manufacture for treating a clotting disorder in a mammal, said article of manufacture comprising a tissue factor composition, wherein said composition comprises tissue factor incorporated into lipid vesicles, and wherein said tissue factor composition, upon administration to a human patient, produces non-thrombogenic levels of thrombin.

23. The article of manufacture of claim 22, further comprising a factor VIIa polypeptide.

24. The article of manufacture of claim 22, further comprising a factor X polypeptide.

25. A composition comprising tissue factor incorporated into lipid vesicles, wherein said vesicles comprise phospholipids linked to a PEG polymer or sphingolipids linked to a PEG polymer.

26. The composition of claim 25, wherein said vesicles contain 0.5 to 50 mol % of said PEG polymer.

27. The composition of claim 25, wherein said PEG polymer has a molecular weight ranging from 500 to 80,000.

28. The composition of claim 25, wherein said PEG polymer has a molecular weight ranging from 20,000 to 40,000.

29. The composition of claim 25, wherein said PEG polymer has a molecular weight ranging from 2,000 to 20,000.

30. The composition of claim 25, wherein said PEG polymer has a molecular weight ranging from 3,000 to 6,000.

31. The composition of claim 25, wherein said phospholipids comprise one or more phospholipids selected from the group consisting of phosphatidylcholine, phosphatidylethanolamine, and phosphatidylserine.

32. The composition of claim 25, wherein said sphingolipids comprise one or more sphingolipids selected from the group consisting of ceramide, sphingomyelins, cerebrosides, and gangliosides.

33. A kit comprising the composition of claim 25.
